(19) United States
(12) Patent Application Publication
(10) Pub. No.: US 2023/0085470 A1
(43) Pub. Date: Mar. 16, 2023

(54) INSERTION DEVICE AND ASSEMBLY COMPRISING SUCH INSERTION DEVICE AND A FLEXIBLE IMPLANTABLE STRIP

(71) Applicants: INSERM (Institut National de la Santé et de la Recherche Médicale), Paris (FR); Université Grenoble Alpes, Saint Martin d'Heres (FR)

(72) Inventors: Mehrdad KHOSH-NEVIS, Gieres (FR); Blaise YVERT, Gieres (FR)

(21) Appl. No.: 17/795,372

(22) PCT Filed: Jan. 29, 2021
(86) PCT No.: PCT/EP2021/052123

§ 371 Date: Jul. 26, 2022

(30) Foreign Application Priority Data
Jan. 30, 2020 (EP) 20154751.0

(51) Int. Cl. A61B 17/34; A61N 1/05

(52) U.S. Cl. CPC A61B 17/3468; A61N 1/05

(57) ABSTRACT

An insertion device is proposed for implanting a flexible implantable strip in the cortex of a living being in a reliable and accurate manner. The insertion device comprises a wire maintained within a lumen of a penetration member so that a free tip portion configured to engage an anchoring hole of the implantable strip protrudes from a distal end of the penetration member Thanks to reduced dimensions of the tip portion of the wire compared to that of the implantable strip, removing the wire from the lumen results in the implantable strip being released from the penetration member while still adhering to the cortex. The wire may be withdrawn by pulling at an end portion opposite to the tip portion. The penetration member can be chosen among a needle and a tube

### TECHNICAL FIELD

The invention relates to an insertion device for implanting a flexible implantable strip in the cortex of a living being, human or animal, and to an assembly comprising such insertion device and a flexible implantable strip.

### BACKGROUND ART

The invention finds applications in neuroscience, neurosurgery and neurology for purposes which rely on a functionality conferred to the implantable strip. For example, the invention may be implemented in neuroscience for research and preclinical study purposes to record intracortical signals with an implantable strip configured accordingly. The invention could however also be implemented in neurosurgery and neurology for diagnosis and/or treatment of disorders to record and/or emit intracortical signals with an implantable strip configured accordingly.

An assembly comprising an implantable strip suitable for recording intracortical signals, and an insertion device is disclosed in document US 2017/0181707.

However, with this known assembly, the implantable strip adheres to the insertion device so that it is withdrawn or at least displaced as the insertion device is withdrawn from the living being. The known assembly then poses a problem of reliability and accuracy in the placement of the implantable strip.

### SUMMARY

The invention aims at solving the above mentioned problems.

To this end, according to an aspect, the invention proposes an insertion device for implanting a flexible implantable strip in the cortex of a living being, the implantable strip being provided with an anchoring hole in an implantable portion, the insertion device comprising:
a penetration member extending along a penetration axis between opposite proximal and distal ends, the penetration member having a lumen extending along the penetration axis and opening axially at the distal end,
a wire configured to be slidably inserted in the lumen, the wire being maintained within the lumen so that a free tip portion protrudes from the distal end of the penetration member,
   wherein the tip portion of the wire is configured to engage the anchoring hole of the implantable strip and the distal end of the penetration member is configured to abut the implantable portion of the implantable strip when the tip portion engages the anchoring hole.

Hence, with the invention, the implantable strip penetrates the cortex with the tip portion of the wire, which tip portion has reduced dimensions compared to that of the implantable strip. Removing the wire from the lumen results in the implantable strip being released from the penetration member while still adhering to the cortex so as to remain where it has been placed even when the insertion device is withdrawn. A reliable and accurate placement of the implantable strip is then ensured.

The wire may have a diameter comprised between 10 μm and 100 μm, preferably between 45 μm and 55 μm, and the tip portion has a length comprised between 0.5 mm and 10 mm, preferably between 2 mm and 5 mm.

The wire may be made of metal, especially tungsten.

The tip portion of the wire may present a free sharpened end.

The penetration member may be configured as a needle, the distal end of the penetration member being beveled.

The penetration member may be configured as a tube, the distal end of the penetration member being rounded.

The lumen may open at the proximal end of the penetration member, the wire extending through the proximal end of the needle.

Thanks to these provisions, the wire may be withdrawn by pulling at an end portion opposite to the tip portion. The distal end of the penetration member then provides an abutment, to which the implantable strip contacts as the wire is withdrawn, enabling the implantable strip to be properly and reliably detached from the insertion device.

The insertion device may further comprise:
a body attached to the penetration member and having an inner space in communication with the lumen of the penetration member, the wire extending within the inner space of the body, and
a retaining element arranged within the inner space of the body and configured to maintain in place a retaining portion of the wire.

According to another aspect, the invention proposes an assembly comprising an insertion device as defined previously, and a flexible implantable strip, the implantable strip being provided with an anchoring hole in an implantable portion.

The implantable strip may comprise at least one microelectrode array.

According to a further aspect, the invention proposes a method for making an insertion device as defined previously, comprising steps of:
providing a penetration member extending along a penetration axis between opposite proximal and distal ends, the penetration member having a lumen extending along the penetration axis and opening axially at the distal end,
providing a wire,
slidably inserting the wire in the lumen, and
maintaining the wire within the lumen so that a free tip portion of the wire protrudes from the distal end of the penetration member, the tip portion of the wire being configured to engage the anchoring hole of the implantable strip and the distal end of the penetration member being configured to abut the implantable portion of the implantable strip when the tip portion engages the anchoring hole.

The wire may have an end portion opposite to the tip portion and the lumen may open at the proximal end of the penetration member, the step of slidably inserting the wire including inserting the end portion of the wire through the distal end of the penetration member.

Still according to a further aspect, the invention proposes a method for preparing an implantation of a flexible implantable strip in the cortex of a living being, the implantable strip being provided with an anchoring hole, the method comprising steps of:
implementing the method for making an insertion device as defined previously,
engaging the tip portion of the wire of the insertion device in the anchoring hole of the implantable strip.

The invention can be implemented in a method for implanting a flexible implantable strip in the cortex of a living being, the implantable strip being provided with an anchoring hole, the method comprising steps of:
implementing the method for preparing an implantation as defined previously,
penetrating the cortex first by the tip portion of the wire and then by the implantable strip,
removing the insertion device from the cortex, the tip portion of the wire being removed from the anchoring hole of the implantable strip, the implantable strip remaining in the cortex.

The wire may have an end portion opposite to the tip portion and the lumen may open at the proximal end of the penetration member, the step of removing the insertion device comprising pulling the wire by the end portion to withdraw the tip portion from the anchoring hole implantable strip.

### BRIEF DESCRIPTION OF DRAWINGS

Other objects and advantages of the invention will emerge from the following disclosure of a particular embodiment of the invention given as a non-limitative example, the disclosure being made in reference to the enclosed drawings in which:

FIG. 1 is a representation of an assembly for implanting a flexible implantable strip in the cortex of a living being, the assembly comprising the flexible implantable strip and an insertion device, the insertion device comprising a penetration member configured as a needle,

FIG. 2 is a representation of an alternative of the insertion device of the assembly of FIG. 1, the penetration member being configured as a tube.

### DESCRIPTION OF EMBODIMENTS

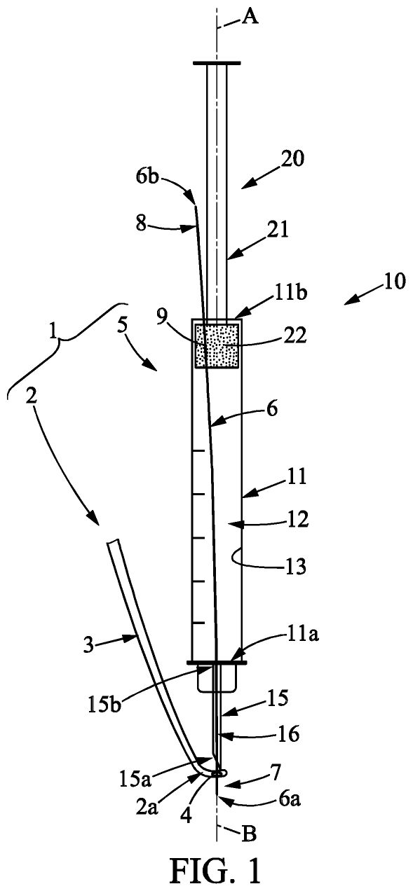

FIG. 1 represents an assembly 1 for implanting a flexible implantable strip 2 in the cortex of a living being, human or animal. The assembly 1 comprises the flexible implantable strip 2 and an insertion device 5.

The implantable strip 2 comprises a flexible substrate 3 provided with an anchoring hole 4 arranged at one end of the substrate 3. The implantable strip also comprises one or several functional members attached to the substrate and configured to confer at least one functionality to the implantable strip 2. Preferably, the functionality is chosen between recording and emitting intracortical signals. In an example, the implantable strip 2 is suitable for recording intracortical signals and comprises one or several microelectrode arrays as functional members. The microelectrode arrays may be arranged on an implantable portion 2a of the implantable strip 2, which implantable portion 2a is intended to be placed within the cortex and extends at least along a length of the implantable strip 2 from the anchoring hole 4. An example of such implantable strip 2 is commercialized by the company Blackrock® Microsystems LLC under the designation MicroFlex Array and disclosed in document US 2017/0181707.

In the represented embodiment, the insertion device 5 comprises a wire 6 and a support member 10 configured to support the wire 6 so that it may remain in a suitable configuration.

In particular, the wire 6 is configured to be the more rigid possible. Such rigidity can be obtained by construction of the wire 6, especially with a suitable material of suitable dimensions and/or through an arrangement of the wire 6 with respect to the support member 10. For example, the wire 6 is made of metal, and especially tungsten, and has a diameter comprised between 10 μm and 100 μm, preferably between 45 μm and 55 μm. The wire 6 extends between opposite tip 7 and end 8 portions. The tip portion 7 is configured to engage the anchoring hole 4 of the implantable strip 2 and may present a free sharpened end 6a whereas the end portion 8 may present an unsharpened end 6b.

In the figures, the support member 10 is in the form of a syringe presenting a syringe axis A. The support member 10 comprises a body 11 extending along the syringe axis A and defining an inner space 12 between a bottom end 11a and opened top end 11b opposite the bottom end 11a.

The support member 10 also comprises a penetration member 15 extending along a penetration axis B between opposite proximal 15b and distal 15a ends.

The proximal end 15b of the penetration member 15 is attached to the bottom end 11a of the body 11 so that the penetration axis B is aligned with, the syringe axis A.

The penetration member 15 has a lumen 16 extending along the penetration axis B and opening along the penetration axis at both the distal 15a and proximal 15b ends. The lumen 16 of the penetration member 15 is then in communication with the inner space 12 of the body 11. The lumen 16 has an inner diameter that is chosen to allow the wire 6 to be slidably inserted therein. Besides, the penetration member 15 has an outer diameter that is chosen not to pass through the anchoring hole 4 of the implantable strip 2. In FIG. 1, although not limited thereto, the penetration member 15 is configured as a needle and the distal end 15a of the penetration member 15 is beveled.

The support member 10 is provided with a retaining element 20 arranged within the inner space 12 of the body 11 and configured to maintain in place the aforementioned wire 6. In the illustrated embodiment, the retaining element 20 is formed by a plunger 21 of the syringe having a piston portion 22 in contact with an inner surface 13 of the body 11.

To make the insertion device 5, the wire 6 is slidably inserted in the lumen 16 of the penetration member 15 until the tip portion 7 freely protrudes from the distal end 15a of the penetration member 15 of a length determined so that the tip portion 7 protruding from the distal end 15a presents an overall rigidity. In the particular embodiment, the end portion 8 of the wire 6 is inserted through the distal end 15a of the penetration member 15 and further through the proximal end 15b of the penetration member 15 within the inner space 12 of the body 11 where a retaining portion 9 of the wire 6 arranged between the tip portion 7 and the end portion 8 is held between the piston portion 22 of the plunger and the inner surface 13 of the body 11. For a wire 6 made of metal, especially tungsten, having a diameter comprised between 10 μm and 100 μm, the tip portion 7 has a length comprised between 0.5 mm and 10 mm, preferably between 2 mm and 5 mm.

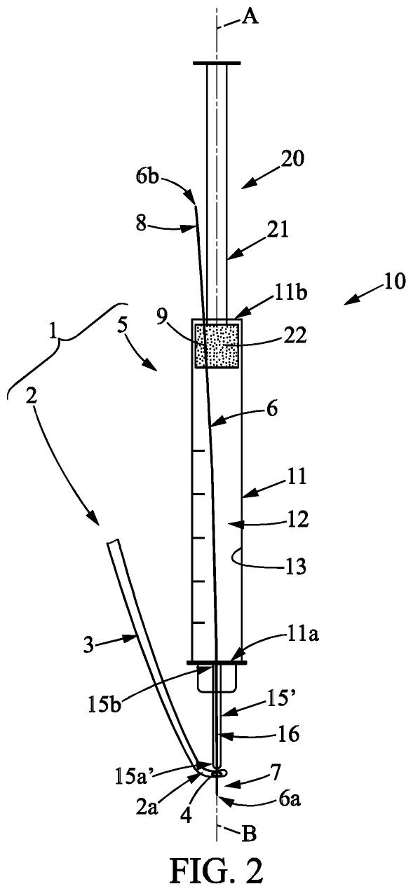

The invention disclosed in relation with a specific embodiment is not limited thereto. In particular, the support member 10 could present some variations. In a variant shown on FIG. 2, the penetration member is configured as a tube and the distal end 15a′ of the penetration member 15′ is rounded rather than beveled. In another variant, the proximal end 15a of the penetration member 15 could be attached to the bottom end 11a of the body 11 so that the penetration axis B is parallel to the syringe axis A but offset from it. Also the support member 10 could present any other suitable configuration than that of a syringe. For example, the support member 10 may have a penetration member 15 with a lumen 16 opened at the sole distal end 15a and retained in any suitable manner so that the tip portion 7 freely protrudes from the distal end 15a.

Once the insertion device 5 has been made as disclosed previously, an implantation of the flexible implantable strip 2 in the cortex of the living being can be prepared by engaging the tip portion 7 of the wire 6 of the insertion device 5 in the anchoring hole 4 of the implantable strip 2.

The assembly 1 can then be implemented to implant the flexible implantable strip 2 in the cortex of the living being.

To that end, the cortex can be penetrated through a hole at its external surface by the tip portion 7 of the wire 6 which engages the anchoring hole 4 of the implantable strip 2.

Once the implantable strip 2 is placed at the desired location with its implantable portion 2a arranged within the cortex, the insertion device 5 may be removed from the cortex. In particular, the tip portion 7 of the wire 6 is first removed from the anchoring hole 4 of the implantable strip 2 by pulling the end portion 8 of the wire 6. The implantable portion 2a of the implantable strip 2 contacts the distal end 15a of the needle 15 providing an abutment to be properly detached from the insertion device 5. The penetration member 15 may then in turn be withdrawn from the living being while the implantable portion 2a of the implantable strip 2 remains in the cortex.

### EXAMPLE 1

An example of an insertion device 5 made in accordance with the teachings of the above disclosure has been tested with the implantable strip 2 MicroFlex Array commercialized by the company Blackrock® Microsystems LLC.

The insertion device 5 comprises:
a 500 μl syringe, with a 30G needle mounted to the body 11, as support member 10, such as the syringe commercialized by the company B-Braun under the denomination Omnican® 50, the body 11 of the syringe being cut in half to reduce the size of the insertion device 5,
a 50-μm-diameter tungsten wire 6 with the sharpened end 6a at the tip portion 7.

The wire 6 is backward inserted into the needle as penetration member 15, of the syringe. The unsharpened end 6b of the end portion 8 of the wire 6 is passed through inside the needle by the distal end 15a and pulled from the opened (cut) top end 11b of the body 11 of the syringe until the sharpened end 6a of the wire exits the distal end 15a of the needle by 2 mm to 3 mm.

The sharpened end 6a of the tip portion 7 of the wire 6 is passed through the anchoring hole 4 located at the end of the implantable strip 2.

The retaining portion 9 of the wire 6 is secured at the back of the syringe using the plunger 21 to prevent the sharpened end 6a of the wire 6 from sinking into the needle during the implantation. The implantable strip 2 is maintained against the distal end 15a of the needle during insertion in the cortex.

A tail of the implantable strip 2 opposite the anchoring hole 4 may be temporarily secured to the back of the syringe in such a way that the implantable strip 2 remains in slight tension preventing the tip portion 7 of the wire 6 to exit the anchoring hole 4 while the insertion device 5 is in a vertical configuration just before the implantation.

The insertion device 5 is installed on a manipulator, perpendicularly (vertically) to a surface of an implantation area, with the needle and the tip portion 7 of the wire 6 facing the surface of the implantation area.

Using the manipulator, the insertion device 5 and consequently the implantable strip 2 are inserted in the cortex.

Once the implantable strip 2 is implanted at a considered depth, the tail of the implantable strip 2 is separated from the insertion device 5. This causes to reduce the adhesion between the needle and the flexible implantable strip 2 while withdrawing the needle. Before withdrawing the needle, the end portion 8 of the wire 6 presenting the unsharpened end 6b is pulled back. In doing so, the sharpened end 6a of the tip portion 7 of the wire 6 enters the needle and releases the implantable strip 2. Withdrawing the needle is carried out gently using a lever (controller) of the manipulator.

The insertion device 5 of example 1 was tested in agarose gel 0.4% and in fresh pig brain. In-vitro tests were performed in wet conditions (to be as similar as possible to real situation during surgery). The success rate of implantation was 90% during 10 tests in agarose gels and 100% success in fresh pig brain. By contrast, a commercial insertion system implemented according to the teachings of document US 201 7/01 81 707 only ensured a success rate of 20% in agarose and of 10-15% in fresh pig brain

### EXAMPLE 2

Another example of an insertion device 5 made in accordance with the teachings of the above disclosure has been tested with the implantable strip 2 MicroFlex Array commercialized by the company Blackrock® Microsystems LLC.

The insertion device 5 comprises:
a 36G non beveled needle, as penetration member, mounted on a 1 mL syringe serving as the body 11, as support member 10, such as the syringe commercialized by the company Terumo under the denomination U-100 Insulin (1 mL) with the needle commercialized by the company Phymep, the body 11 of the syringe being cut in half to reduce the size of the insertion device 5,
a 50-μm-diameter tungsten wire 6 with the sharpened end 6a at the tip portion 7.

Mounting of the wire 6 and the implantable strip 2 as well as implantation of is performed as disclosed previously in relation with example 1.

The insertion device 5 of example 2 was tested:
in-vitro in agarose gel 0.4%,
ex-vivo in fresh pig brain, and
in-vivo in real surgeries in 3 minipigs.

In-vitro tests were performed in wet conditions (to be as similar as possible to real situation during surgery). The success rate of implantation was 90% during 10 tests in agarose gels and 100% success in fresh pig brain. During the implantation in real surgeries, the success rate was more than 90%. By contrast, the commercial insertion system implemented according to the teachings of document US 201 7/01 81 707 only ensured a success rate of 20% in agarose, of 10-15% in fresh pig brain and 10-15% in in-vivo implantations.

1. Insertion device for implanting a flexible implantable strip in the cortex of a living being, the flexible implantable strip being provided with an anchoring hole in an implantable portion, the insertion device comprising:
a penetration member extending along a penetration axis between opposite proximal and distal ends, the penetration member having a lumen extending along the penetration axis and opening axially at the distal end
a wire configured to be slidably inserted in the lumen, the wire being maintained within the lumen so that a free tip portion protrudes from the distal end of the penetration member,
wherein the free tip portion of the wire is configured to engage the anchoring hole of the implantable strip and the distal end of the penetration member is configured to abut the implantable portion of the flexible implantable strip when the free tip portion engages the anchoring hole.

2. Insertion device according to claim 1, wherein the wire has a diameter comprised between 10 μm and 100 μm, and the free tip portion has a length comprised between 0.5 mm and 10 mm.

3. Insertion device according to claim 1, wherein the wire is made of metal.

4. Insertion device according to claim 1, wherein the free tip portion of the wire presents a free sharpened end.

5. Insertion device according to claim 1, wherein the penetration member is configured as a needle, the distal end of the penetration member being beveled.

6. Insertion device according to claim 1, wherein the penetration member is configured as a tube, the distal end of the penetration member being rounded.

7. Insertion device according to claim 1, wherein the lumen opens at the proximal end of the penetration member, the wire extending through the proximal end of the penetration member.

8. Insertion device according to claim 7, wherein the insertion device further comprises:
a body attached to the penetration member and having an inner space in communication with the lumen of the penetration member, the wire extending within the inner space of the body, and
a retaining element arranged within the inner space of the body and configured to maintain in place a retaining portion of the wire.

9. Assembly comprising an insertion device according to claim 1, and a flexible implantable strip, the flexible implantable strip being provided with an anchoring hole in an implantable portion.

10. Assembly according to claim 9, wherein the flexible implantable strip comprises at least one microelectrode array.

11. Method for making an insertion device according claim 1, comprising steps of:
providing a penetration member extending along a penetration axis between opposite proximal and distal ends, the penetration member having a lumen extending along the penetration axis and opening axially at the distal end,
providing a wire,
slidably inserting the wire in the lumen, and
maintaining the wire within the lumen so that a free tip portion of the wire protrudes from the distal end of the penetration member, the free tip portion of the wire being configured to engage the anchoring hole of the flexible implantable strip and the distal end of the penetration member being configured to abut the implantable portion of the flexible implantable strip when the free tip portion engages the anchoring hole.

12. Method according to claim 11, wherein the wire has an end portion opposite the free tip portion and the lumen opens at the proximal end of the penetration member, and wherein the step of slidably inserting the wire includes inserting the end portion of the wire through the distal end of the penetration member.

13. Method for preparing an implantation of a flexible implantable strip in the cortex of a living being, the flexible implantable strip being provided with an anchoring hole, the method comprising steps of:
implementing the method for making an insertion device according to claim 11, and
engaging the free tip portion of the wire of the insertion device in the anchoring hole of the flexible implantable strip.

14. Insertion device according to claim 2, wherein the wire has a diameter from 45 μm to 55 μm.

15. Insertion device according to claim 2, wherein the free tip portion has a length comprised from 2 mm to 5 mm.

16. The insertion device according to claim 3, wherein the metal is tungsten.
